(19) United States
(12) Patent Application Publication
(10) Pub. No.: US 2018/0280279 A1
(43) Pub. Date: Oct. 4, 2018

(54) SOAP AND SURFACTANT FREE SHAVE LOTION COMPOSITION

(71) Applicant: L'OREAL, Paris (FR)

(72) Inventors: William R. FEUER, Hillsborough, NJ (US); Wivina LIJO, Flemington, NJ (US)

(21) Appl. No.: 15/476,393

(22) Filed: Mar. 31, 2017

(51) Int. Cl. A61K 8/81; A61K 8/86; A61K 8/02; A61K 8/37; A61Q 9/02; A61K 8/89; A61K 8/97; A61K 8/67

(52) U.S. Cl. CPC A61K 8/8152; A61K 8/86; A61K 8/0216; A61K 8/37; A61K 2800/33; A61K 8/89; A61K 8/97; A61K 8/67; A61Q 9/02

(57) ABSTRACT

A cosmetic composition and a method of preparing a cosmetic composition are provided. The cosmetic composition includes (a) at least one water soluble resin, (b) at least one emulsifying polymer, (c) aqueous phase, and (d) oil phase. The cosmetic composition is soap-free and surfactant-free.

### FIELD OF THE INVENTION

The present invention relates to a cosmetic composition. More particularly, the invention relates to a soap and surfactant free shave lotion composition having at least one water soluble resin, at least one emulsifying polymer, aqueous phase, and oil phase.

### BACKGROUND OF THE INVENTION

Conventional self-foaming shaving lotions are valued by a wide range of consumers. However, these products, due to the presence of soap or surfactant in the composition, have a tendency to dry out the skin and to increase the roughness of the skin. To mitigate this effect, self-foaming shaving lotions have been formulated with softening agents such as humectants, emollients, silicones, and the like. The incorporation of these additives has an influence on the aesthetic qualities of the product and may also cause dryness of the skin by repeated use. Furthermore, users have to rinse off shaving lotions after shaving to remove residual soap or surfactant. For these reasons “soap and surfactant free” shaving lotion has been developed in this invention.

### BRIEF SUMMARY OF THE INVENTION

In an exemplary embodiment, a cosmetic composition is provided. The composition includes at least one water soluble resin, at least one emulsifying polymer, an aqueous phase, an oil phase. The cosmetic composition is soap-free and surfactant-free.

In another exemplary embodiment, a cosmetic composition is provided. The cosmetic composition includes about 0.1 to about 1% of at least one water soluble resin, about 0.1 to about 2% at least one emulsifying polymer, about 70 to about 99% aqueous phase, and about 1 to about 30% oil phase. The cosmetic composition is soap-free and surfactant-free.

In another exemplary embodiment, a cosmetic composition is provided. The cosmetic composition includes about 0.1 to about 1% of at least one water soluble resin including PEG-14M, about 0.1 to about 2% at least one emulsifying polymer selected from ammonium acryloyldimethyltaurate/steareth-25 methacrylate crosspolymer and ammonium acryloyldimethyltaurate/vp copolymer, about 70 to about 99% aqueous phase including water, about 1 to about 30% oil phase including caprylic/capric triglyceride, and optional components selected from the group consisting of actives, fragrance, preservatives, and combinations thereof. The cosmetic composition is soap-free and surfactant-free.

In another exemplary embodiment, a method of preparing a cosmetic composition is provided. The cosmetic composition includes at least one water soluble resin, at least one emulsifying polymer, an aqueous phase, an oil phase. The cosmetic composition is soap-free and surfactant-free.

### DETAILED DESCRIPTION OF THE INVENTION

Other than in the operating examples, or where otherwise indicated, all numbers expressing quantities of ingredients and/or reaction conditions are to be understood as being modified in all instances by the term “about,” meaning within 10% of the indicated number (e.g. “about 10%” means 9%-11% and “about 2%” means 1.8%-2.2%).

All percentages and ratios are calculated by weight unless otherwise indicated. All percentages are calculated based on the total composition unless otherwise indicated. Generally, unless otherwise expressly stated herein, “weight” or “amount” as used herein with respect to the percent amount of an ingredient refers to the amount of the raw material comprising the ingredient, wherein the raw material may be described herein to comprise less than and up to 100% activity of the ingredient. Therefore, weight percent of an active in a composition is represented as the amount of raw material containing the active that is used, and may or may not reflect the final percentage of the active, wherein the final percentage of the active is dependent on the weight percent of active in the raw material.

It should be understood that the precise numerical values used in the specification, including the examples and claims, form additional embodiments of the invention, and are intended to include any ranges which can be narrowed to any to end points disclosed within the exemplary ranges and values provided. Efforts have been made to ensure the accuracy of the numerical values disclosed. However, any measured value can inherently contain certain errors resulting from the standard deviation found in its respective measuring technique.

The term “comprising” (and its grammatical variations) as used herein is used in the inclusive sense of “having” or “including” and not in the exclusive sense of the terms “consisting only of,” “consisting essentially of” and “consisting of.”

“At least one,” as used herein, means one or more and thus includes individual components as well as mixtures/combinations.

The articles “a” and “an,” as used herein, mean one or more when applied to any feature in embodiments of the present invention described in the specification and claims. The use of “a” and “an” does not limit the meaning to a single feature unless such a limit is specifically stated. The article “the” preceding singular or plural nouns or noun phrases denotes a particular specified feature or particular specified features and may have a singular or plural connotation depending upon the context in which it is used. The adjective “any” means one, some, or all indiscriminately of whatever quantity.

The term “cosmetic composition” is understood to mean a composition compatible with keratinous substances, which exhibits a pleasant color, odor and feel and which does not cause unacceptable discomfort liable to turn the consumer away from it.

The term “shaving composition” is understood to mean a composition compatible with keratinous substances, which is particularly formulated to help removal or cutting of hair including facial hair, head hair, chest hair, abdominal hair, leg hair, underarm hair, pubic hair, or any other bodily hair.

The term “soap” is defined, in accordance with the invention, as salts of carboxylic fatty acids.

The term “soap-free” means that the composition contains no soap.

The term “surfactant” is defined, in accordance with the invention, as a compound having at least one hydrophilic moiety and at least one lipophilic moiety.

The term “surfactant-free” means that the composition contains no surfactant.

The compositions and methods of the present invention can comprise, consist of, or consist essentially of the essential elements and limitations of the invention described herein, as well as any additional or optional ingredients, components, or limitations described herein or otherwise useful.

Unless otherwise specified herein, all percentages and ratios of components are by weight relative to the total weight of the final composition.

Provided are exemplary a cosmetic composition and a method of preparing a cosmetic composition. Embodiments of the present disclosure, in comparison to methods and articles not utilizing one or more features disclosed herein, unexpectedly provide for a close shave, with visual and tactile skin smoothness that is improved over, or at least statistically equivalent to, conventional shave products that include soaps and surfactants. The compositions of the instant disclosure avoid the skin drying, tackiness, and irritation associated with soap or surfactants, and avoid the need to be rinsed off the skin, and also enable non-clogging of razor to confer an excellent glide during shaving.

In an embodiment, a cosmetic composition is provided. The composition may comprise (a) at least one water soluble resin, (b) at least one emulsifying polymer, (c) aqueous phase, and (d) oil phase. The cosmetic composition is soap-free and surfactant-free.

In an embodiment, a cosmetic composition is provided. The composition may consist of (a) at least one water soluble resin, (b) at least one emulsifying polymer, (c) aqueous phase, and (d) oil phase.

In another embodiment, a cosmetic composition is provided. The cosmetic composition may comprise (a) about 0.1 to about 1% of at least one water soluble resin, (b) about 0.1 to about 2% at least one emulsifying polymer, (c) about 70 to about 99% aqueous phase, and (d) about 1 to about 30% oil phase. The cosmetic composition is soap-free and surfactant-free.

In another embodiment, a cosmetic composition is provided. The cosmetic composition may consist of (a) about 0.1 to about 1% of at least one water soluble resin, (b) about 0.1 to about 2% at least one emulsifying polymer, (c) about 70 to about 99% aqueous phase, and (d) about 1 to about 30% oil phase.

In another embodiment, a cosmetic composition is provided. The cosmetic composition may comprise (a) about 0.1 to about 1% of at least one water soluble resin including PEG-14M, (b) about 0.1 to about 2% at least one emulsifying polymer including ammonium acryloyldimethyltaurate/steareth-25 methacrylate crosspolymer and ammonium acryloyldimethyltaurate/vp copolymer, (c) about 70 to about 99% aqueous phase including water, and (d) about 1 to about 30% oil phase including caprylic/capric triglyceride, and optional components selected from the group consisting of actives, fragrance, preservatives, and combinations thereof. The cosmetic composition is soap-free and surfactant-free.

In another embodiment, a cosmetic composition is provided. The cosmetic composition may consist of (a) about 0.1 to about 1% of at least one water soluble resin including PEG-14M, (b) about 0.1 to about 2% at least one emulsifying polymer including ammonium acryloyldimethyltaurate/steareth-25 methacrylate crosspolymer and ammonium acryloyldimethyltaurate/vp copolymer, (c) about 70 to about 99% aqueous phase including water, and (d) about 1 to about 30% oil phase including caprylic/capric triglyceride, and optional components selected from the group consisting of actives, fragrance, preservatives, and combinations thereof. The cosmetic composition is soap-free and surfactant-free.

In another embodiment, a method of preparing a cosmetic composition is provided. The cosmetic composition includes at least one water soluble resin, at least one emulsifying polymer, aqueous phase, oil phase. The cosmetic composition is soap-free and surfactant-free.

The cosmetic composition may be in the form of cream, paste, or gel

Water Soluble Resin

In one embodiment, the water soluble resin is selected from the group consisting of PEG-5M, PEG-7M, PEG-9M, PEG-14M, PEG-20M, PEG-23M, PEG-45M, PEG-90M, and combinations thereof. The water soluble resin may be selected from any other family or derivative of PEG compounds.

In an exemplary embodiment, a useful water soluble for use in the present invention is PEG-14M sold under the tradename POLYOX™ WSR 205 Water Soluble Resin by Dow Chemical.

The water soluble resin of the present invention is typically employed in an amount ranging from about 0.1% to about 1%, from about 0.2% to about 0.9%, from about 0.3% to about 0.8%, from about 0.4% to about 0.7%, from about 0.5% to about 0.6% or any suitable combination, sub-combination, range, or sub-range thereof by weight, based on the total weight of the composition. In an embodiment, the water soluble resin of the present invention is typically employed in an amount not more than 1% by weight, based on the total weight of the composition

Emulsifying Polymer

In one embodiment, the emulsifying polymer is selected from the group consisting of ammonium acryloyldimethyltaurate/steareth-25 methacrylate crosspolymer, ammonium acryloyldimethyltaurate/vp copolymer, ammonium acryloyldimethyltaurate/beheneth-25 methacrylate crosspolymer, acrylates/ceteth-20 itaconate copolymer, and combinations thereof.

In an exemplary embodiment, emulsifying polymer for use in the present invention is selected from ammonium acryloyldimethyltaurate/steareth-25 methacrylate crosspolymer and ammonium acryloyldimethyltaurate/vp copolymer. In another embodiment, the emulsifying polymer includes both ammonium acryloyldimethyltaurate/steareth-25 methacrylate crosspolymer and ammonium acryloyldimethyltaurate/vp copolymer. In an embodiment, the emulsifying polymer consists of both ammonium acryloyldimethyltaurate/steareth-25 methacrylate crosspolymer and ammonium acryloyldimethyltaurate/vp copolymer.

In some examples, the emulsifying polymer is available in the formulations under the indicated trade names, including Seppic: Sepigel 305, Simulgel 600, Simulgel INS100, Sepiplus 265, Sepilplus 400, Sepinov EMT10, Sepimax Zen. From Lubrizol Pemulen TR-1, Pemulen TR-2.

The emulsifying polymer of the present invention is typically employed in an amount ranging from about 0.1% to about 2%, from about 0.2% to about 1.9%, from about 0.3% to about 1.8%, from about 0.4% to about 1.7%, from about 0.5% to about 1.6%, from about 0.6% to about 1.5%, from about 0.7% to about 1.4%, from about 0.8% to about 1.3%, from about 0.9% to about 1.2%, from about 1.0% to about 1.1% by weight, or any suitable combination, sub-combination, range, or sub-range thereof by weight, based on the total weight of the composition. In an embodiment, the emulsifying polymer of the present invention is typically employed in an amount not more than 2% by weight, based on the total weight of the composition.

Emulsifying polymer provides the “quick breaking” effect so that good surface contact can be formed between the composition and the skin. The “quick breaking effect” also becomes clear on application which allows users to shave in detail around bears and moustaches. The composition becomes thin on viscosity which prevents a razor from getting clogged during shaving

Aqueous Phase

In the various embodiments, the aqueous phase includes water and other components selected from the group consisting of water, humectants, other optional components and combinations thereof. The humectants may include glycerin, sorbitol, pentaerythritol, inositol, pyrrolidone carboxylic acid, and combinations thereof.

The humectant is typically employed in an amount ranging from about 1% to about 20%, from about 5% to about 15%, about 10% by weight, or any suitable combination, sub-combination, range, or sub-range thereof by weight, based on the total weight of the composition.

In an exemplary embodiment, humectant for use in the present invention is glycerin.

The aqueous phase of the present invention is typically employed in an amount ranging from about 70% to about 99%, from about 75% to about 95%, from about 80% to about 90%, or any suitable combination, sub-combination, range, or sub-range thereof by weight, based on the total weight of the composition.

Oil Phase

In the various embodiments, the oil phase includes a component selected from the group consisting of cyclomethicone, silicone oil such as dimethicone, dicapryl maleate, caprylic/capric triglyceride, isopropyl myristate, octyl stearate, isostearyl linoleate, botanical oil, lanolin oil, coconut oil, cocoa butter, olive oil, avocado oil, aloe extracts, jojoba oil, castor oil, aleurites moluccanus seed oil, fatty acid, oleic acid, stearic acid, fatty alcohol, cetyl alcohol, hexadecyl alcohol, diisopropyl adipate, hydroxybenzoate esters, benzoic acid esters of C₉-C₁₅ alcohols, isononyl iso-nonanoate, alkanes, mineral oil, silicone, dimethyl polysiloxane, ether, polyoxypropylene butyl ether, polyoxypropylene cetyl ether, C₁₂-C₁₅ alkyl benzoate, aryl alkyl benzoate, isopropyl lauroyl sarcosinate, and combinations of these.

In an exemplary embodiment, an oil phase component for use in the present invention is selected from one or a combination of caprylic/capric triglyceride, dimethicone, jojoba oil, and aleurites moluccanus seed oil (providing benefits also as a skin calming agent).

The oil phase of the present invention is typically employed in an amount ranging from about 1% to about 30%, from about 5% to about 25%, from about 10% to about 20%, about 15% by weight, or any suitable combination, sub-combination, range, or sub-range thereof by weight, based on the total weight of the composition.

Optional Components

In one embodiment, the composition may include optional components selected from the group consisting of actives, fragrance, preservatives, and combinations thereof. The actives are selected from the group consisting of butylated hydroxytoluene, tocopherol, tocopheropl derivatives, tocotrienol, tocotrienol derivatives, ascorbic acid, ascorbic acid derivatives, ascorbyl palmitate, vitamin E, vitamin C, and combinations thereof. The preservatives are selected from the group consisting of phenoxyethanol, caprylyl glycol, and combinations thereof.

In some exemplary embodiments, optional components included in the inventive formulations are selected from vitamin E and derivatives, aloe vera, allantoin, and combinations of these.

The actives of the present invention is typically employed in an amount ranging from about 0.01% to about 1%, from about 0.1% to about 0.9%, from about 0.2% to about 0.8%, from about 0.3% to about 0.7%, from about 0.4% to about 0.6%, about 0.5%, or any suitable combination, sub-combination, range, or sub-range thereof by weight, based on the total weight of the composition.

The above ingredients lists are only examples and not limiting.

The compositions according to the instant disclosure may be prepared according to techniques that are well known to those skilled in the art, in particular those intended for the preparation of shaving compositions.

The instant disclosure will be better understood from the examples that follow, all of which are intended for illustrative purposes only and are not meant to limit the scope of the instant disclosure in any way.

### Examples

TABLE 1
Inventive Example

| Phase | US INCI name | Inventive |
|---|---|---|
| A | HUMECTANT | 1-20% |
| | PRESERVATIVE | 0.5-1% |
| | SOLVENT | 0.1-1% |
| | WATER | balance |
| | PEG-14M | 0.1-1% |
| B | AMMONIUM | 0.1-0.5% |
| | ACRYLOYLDIMETHYLTAURATE/STEARETH-25 | |
| | METHACRYLATE CROSSPOLYMER | |
| | AMMONIUM ACRYLOYLDIMETHYLTAURATE/VP | |
| | COPOLYMER | 0.5-1.0% |
| C | VITAMIN | 0.05-0.15% |
| | FRAGRANCE | 0.1-0.3% |
| | CAPRYLIC/CAPRIC TRIGLYCERIDE | 1-30% |
| | OIL (botanical) (I) | 0.5-1.5% |
| | OIL (botanical) (II) | 0.1-0.5% |
| D | SILICONE OIL | 1-5% |
| | Optional Additives | <1% |
| | TOTAL | 100% |

Raw Materials:

PEG-14M (POLYOX WSR 205 AMERCHOL, 100% active, from DOW CHEMICAL; a polymer of ethylene oxide that conforms generally to the formula: H(OCH₂CH₂)ₙOH) where n has an average value of 14000); AMMONIUM ACRYLOYLDIMETHYLTAURATE/STEARETH-25 METHACRYLATE CROSSPOLYMER (ARISTOFLEX HMS, 100% active, from CLARIANT; a copolymer of ammonium acryloyldimethyltaurate and steareth-25 methacrylate monomers)

The Inventive in Table 1 may be prepared according to the procedure as follows: The aqueous phase, Phase A ingredients are added, one at a time, to a main mixing tank, starting with water, with continuous mixing, followed by heating to ensure complete hydration of PEG-14M. Phase B ingredients are added and mixed slowly until uniform. In a separate mixing tank, Phase C ingredients are added, one at a time and mixed until uniform. With a side sweep mixer and an homogenizer, the Phase C ingredients are added to the main tank for mixing, followed by the Phase D ingredients, after which addition the mixture is side sweep mixed and homogenized and for a further 30 minutes, while cooling.

Trials with Inventive Formulations Vs Conventional Surfactant/Soap Based Foaming Shave Product.

A group of 30 male subjects were selected to participate in the evaluation of the inventive formulation shaving product (“Inventive”) verses a commercially available comparative, Edge® Shaving Gel (“Comparative” and a product sold under registered ™ to Edgewell Personal Care Brands, LLC). The study included clinical grading of facial skin smoothness. Subjects applied Inventive to one side of the face and Comparative to the opposite side, followed by shaving with a disposable razor. Treatment assignment to the right or left side of the face was determined by a computer-generated randomization schedule. Visual and tactile facial smoothness were assessed on each side of the face by an expert grader before treatment (Baseline) and following treatment (Immediate). The same grader was used throughout the study. The following scales were used:

Skin Texture/Smoothness (Tactile); scale from 0 1 2 3 4 5 6 7 8 to 9, where: 0=Smooth, soft even skin texture/no beard stubble; 1-3=Mild Roughness/minimal stubble; 4-6=Moderate Roughness and Stubble; and 7-9=Rough, uneven skin texture/coarse beard.

Skin Texture/Smoothness (Visual); scale from 0 1 2 3 4 5 6 7 8 to 9, where: 0=Smooth, soft skin appearance; 1-3=Mild roughness; 4-6=Moderate roughness; and 7-9=Rough, uneven skin appearance.

Half-point scores were used to discriminate midpoint values.

Results: At Baseline, Subject skin receiving Inventive had a significantly better Skin Texture/Smoothness (Tactile) scores than Comparative, thus ANCOVA with baseline value as covariate was used for comparing the difference in change from baseline for Skin Texture/Smoothness (Tactile) between the two treatments.

Under the conditions of this study and in this population, statistically significant improvements in visual and tactile skin texture/smoothness were observed following treatment with both Inventive and Comparative, and improvements in visual and tactile skin texture/smoothness were statistically equivalent between treatments. The results show that the inventive foaming shave products that lack soap and surfactants are at least as effective as comparative in providing desirable post treatment texture and skin smoothness.

While the invention has been described with reference to a preferred embodiment, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims.

1. A cosmetic composition comprising:
(a) at least one water soluble resin;
(b) at least one emulsifying polymer;
(c) aqueous phase; and
(d) oil phase,
wherein:
the cosmetic composition is soap-free and surfactant-free;
the cosmetic composition is a shaving composition; and
the cosmetic composition includes a quick breaking effect, wherein, upon application, the composition becomes clear and thin on viscosity, increasing visibility of details around beards and moustaches, and preventing razor clogging during shaving.

2. The composition of claim 1, wherein said at least one water soluble resin is selected from the group consisting of PEG-5M, PEG-7M, PEG-9M, PEG-14M, PEG-20M, PEG-23M, PEG-45M, PEG-90M, and combinations thereof.

3. The composition of claim 1, wherein said at least one emulsifying polymer is selected from the group consisting of ammonium acryloyldimethyltaurate/steareth-25 methacrylate crosspolymer, ammonium acryloyldimethyltaurate/vp copolymer, ammonium acryloyldimethyltaurate/beheneth-25 methacrylate crosspolymer, acrylates/ceteth-20 itaconate copolymer, and combinations thereof.

4. The composition of claim 1, wherein said aqueous phase includes water.

5. The composition of claim 1, wherein said aqueous phase includes humectants selected from the group consisting of glycerin, sorbitol, pentaerythritol, inositol, pyrrolidone carboxylic acid, and combinations thereof.

6. The composition of claim 1, wherein said oil phase is selected from the group consisting of cyclomethicone, dimethicone, dicapryl maleate, caprylic/capric triglyceride, isopropyl myristate, octyl stearate, isostearyl linoleate, botanical oil, lanolin oil, coconut oil, cocoa butter, olive oil, avocado oil, aloe extracts, jojoba oil, castor oil, fatty acid, oleic acid, stearic acid, fatty alcohol, cetyl alcohol, hexadecyl alcohol, diisopropyl adipate, hydroxybenzoate esters, benzoic acid esters of C₉-C₁₅ alcohols, isononyl iso-nonanoate, alkanes, mineral oil, silicone, dimethyl polysiloxane, ether, polyoxypropylene butyl ether, polyoxypropylene cetyl ether, C₁₂-C₁₅ alkyl benzoate, aryl alkyl benzoate, isopropyl lauroyl sarcosinate, and combinations thereof.

7. The composition of claim 1, wherein said at least one water soluble resin is present in an amount of about 0.1 to about 1% by weight based on the total weight of the composition.

8. The composition of claim 1, wherein said at least one emulsifying polymer is present in an amount of about 0.1 to about 2% by weight based on the total weight of the composition.

9. The composition of claim 1, wherein said aqueous phase is present in an amount of about 70 to about 99% by weight based on the total weight of the composition.

10. The composition of claim 1, wherein said oil phase is present in an amount of about 10 to about 30% by weight based on the total weight of the composition.

11. The composition of claim 1, further comprising actives selected from the group consisting of butylated hydroxytoluene, tocopherol, tocopherol derivatives, tocotrienol, tocotrienol derivatives, ascorbic acid, ascorbic acid derivatives, ascorbyl palmitate, vitamin E, vitamin C, and combinations thereof.

12. The composition of claim 1, further comprising fragrance.

13. The composition of claim 1, further comprising preservatives selected from the group consisting of phenoxyethanol, caprylyl glycol, and combinations thereof.

14. The cosmetic composition of claim 1, wherein the cosmetic composition is in the form of cream, paste, or gel.

15. A cosmetic composition comprising:
(a) about 0.1 to about 1% of at least one water soluble resin;
(b) about 0.1 to about 2% at least one emulsifying polymer;
(c) about 70 to about 99% aqueous phase; and
(d) about 1 to about 30% oil phase,
wherein:
all amounts are percentages by weight based on the total weight of the composition;
the cosmetic composition is soap-free and surfactant-free;
the cosmetic composition is a shaving composition; and
the cosmetic composition includes a quick breaking effect, wherein, upon application, the composition becomes clear and thin on viscosity, increasing visibility of details around beards and moustaches, and preventing razor clogging during shaving.

16. A cosmetic composition comprising:
(a) about 0.1 to about 1% of at least one water soluble resin including PEG-14M;
(b) about 0.1 to about 2% at least one emulsifying polymer including ammonium acryloyldimethyltaurate/steareth-25 methacrylate crosspolymer and ammonium acryloyldimethyltaurate/vp copolymer;
(c) about 70 to about 99% aqueous phase including water;
(d) about 1 to about 30% oil phase including caprylic/capric triglyceride; and
(e) optional components selected from the group consisting of actives, fragrance, preservatives, and combinations thereof,
wherein:
all amounts are percentages by weight based on the total weight of the composition;
the cosmetic composition is soap-free and surfactant-free;
the cosmetic composition is a shaving composition; and
the cosmetic composition includes a quick breaking effect, wherein, upon application, the composition becomes clear and thin on viscosity, increasing visibility of details around beards and moustaches, and preventing razor clogging during shaving.

17. The composition of claim 16, wherein said actives are selected from the group consisting of butylated hydroxytoluene, tocopherol, tocopheropl derivatives, tocotrienol, tocotrienol derivatives, ascorbic acid, ascorbic acid derivatives, ascorbyl palmitate, vitamin E, vitamin C, and combinations thereof.

18. The composition of claim 16, wherein said preservatives are selected from the group consisting of phenoxyethanol, caprylyl glycol, and combinations thereof.

19. A method of preparing a cosmetic composition comprising mixing:
(a) at least one water soluble resin;
(b) at least one emulsifying polymer;
(c) aqueous phase; and
(d) oil phase,
wherein:
the cosmetic composition is soap-free and surfactant-free;
the cosmetic composition is a shaving composition; and
the cosmetic composition includes a quick breaking effect, wherein, upon application, the composition becomes clear and thin on viscosity, increasing visibility of details around beards and moustaches, and preventing razor clogging during shaving.

20. (canceled)

21. (canceled)
